(19) United States
(12) Patent Application Publication
(10) Pub. No.: US 2025/0295351 A1
(43) Pub. Date: Sep. 25, 2025

(54) Concussion Sensor

(71) Applicant: Phreno Inc., Denver, CO (US)

(72) Inventors: Carter Grinnell, Golden, CO (US); Nicholas Kasten, Denver, CO (US)

(73) Assignee: Phreno Inc., Denver, CO (US)

(21) Appl. No.: 19/084,746

(22) Filed: Mar. 19, 2025

Related U.S. Application Data
(60) Provisional application No. 63/567,153, filed on Mar. 19, 2024.

(51) Int. Cl. A61B 5/00; A42B 3/04; A61B 5/11

(52) U.S. Cl. CPC A61B 5/4064; A42B 3/046; A61B 5/11; A61B 5/6803; A61B 5/7203; A61B 5/7267; A61B 5/7275; A61B 5/742; A61B 5/746; A61B 2560/0209; A61B 2562/0219

(57) ABSTRACT

The present invention is a wearable concussion detection and monitoring system that captures and analyzes head impact data in real time. It integrates an accelerometer, a gyroscope, and a microcontroller with Bluetooth Low Energy (BLE) for wireless communication. When an impact exceeds a threshold, the system records linear and rotational acceleration, applies sensor fusion algorithms (e.g., Madgwick filter) to remove gravitational noise, and timestamps the event. Data is stored onboard and transmitted to a mobile application and cloud platform for further analysis. Machine learning models assess concussion risk, providing real-time alerts and long-term impact tracking for athletes, trainers, and medical professionals. The system enhances concussion assessment and injury prevention across sports, military, and other high-impact activities, offering an objective, data-driven approach to head injury monitoring.

### FIELD OF THE INVENTION

The present invention relates to systems and methods for detecting, recording, and analyzing head impacts that may lead to concussions. Specifically, the invention utilizes a multi-sensor approach integrating high-G accelerometers, gyroscopic motion detection, and wireless data transmission for real-time concussion risk assessment. The present invention pertains to a concussion sensor that is able to register impacts surrounding one's head throughout activities such as, but not limited to, football, alpine skiing, lacrosse, downhill mountain biking, soccer, etc. This sensor is not exclusive to a certain discipline and can be used broadly, as long as the safety/mounting requirements are adhered to.

### BACKGROUND OF THE INVENTION

Concussions and other traumatic brain injuries (TBIs) pose a significant risk in contact sports, military activities, and other high-impact environments. Existing concussion detection methods rely on subjective assessments or post-incident evaluations. There is a need for a real-time, sensor-based solution that provides objective, quantifiable data to aid in immediate medical decision-making.

Existing solutions, such as found in U.S. Pat. No. 9,247,780 to Iuliano et al., incorporated herein in its entirety by reference for all purposes, titled Accessory with Integrated Impact Detection Device, describes a protective accessory designed for helmets, integrating an impact detection device. The accessory attaches rigidly to the helmet's external shell using compatible fasteners. The integrated impact detection device is secured within the protective element, ensuring it remains in place during use. This design aims to enhance user safety by providing reliable impact monitoring without compromising the helmet's structural integrity.

Existing solutions, such as found in Canadian Patent No. 2,820,641 to Evans et al., incorporated herein in its entirety by reference for all purposes, titled Impact Sensing Device and Helmet Incorporating the Same, details an impact sensing device equipped with multiple accelerometers oriented orthogonally to each other, allowing for precise detection of impact magnitude and direction. The device can be attached to various body locations and is configured to activate an indicator when an impact exceeds a predetermined threshold. Additionally, the patent discusses the integration of this impact sensing device into helmets, enhancing head protection by providing immediate feedback on significant impacts.

Existing solutions, such as found in U.S. Pat. No. 9,717,457 to Iuliano et al., incorporated herein in its entirety by reference for all purposes, titled Impact Detection System for Protective Headgear. This patent introduces an impact detection system specifically designed for protective headgear. The system includes sensors capable of measuring both linear and rotational accelerations, providing comprehensive data on impacts. It features wireless communication capabilities, allowing real-time transmission of impact data to external devices for monitoring and analysis. The system aims to improve safety in activities where head injuries are a concern by enabling prompt responses to significant impacts.

Existing solutions, such as found in U.S. Pat. No. 11,980,247 to Velasco, incorporated herein in its entirety by reference for all purposes, titled Helmet with Integrated Impact Sensor and Communication System presents a helmet integrated with an impact sensor and a communication system. The impact sensor detects forces exerted on the helmet, while the communication system transmits this data to a remote device for analysis. The integration aims to provide immediate information regarding potential head injuries, facilitating timely medical evaluations and enhancing user safety in environments prone to head impacts.

Existing solutions, such as found in U.S. Pat. No. 9,070,269 to Evans et al., incorporated herein in its entirety by reference for all purposes, titled System and Method for Monitoring Impacts to a Sports Player outlines a system and method for monitoring impacts sustained by sports players. It involves sensors attached to the player's equipment, which detect and measure impact forces. The collected data is transmitted wirelessly to a monitoring system that analyzes the impacts in real-time. The system aims to identify potentially harmful impacts promptly, allowing for immediate intervention and management to protect the athlete's health and safety.

Existing solutions, such as found in U.S. Pat. No. 5,621,922 to Rush, incorporated herein in its entirety by reference for all purposes, titled Sports Helmet Capable of Sensing Linear and Rotational Forces describes a signaling device integrated into headwear, such as athletic helmets, equipped with sensors to detect both linear and rotational impacts that exceed a predetermined magnitude. Upon detecting such an impact, the sensors trigger the signaling device to emit a perceivable alert, notifying observers that a potentially injurious event has occurred. The invention aims to enhance safety by providing immediate awareness of significant impacts, facilitating prompt assessment and response.

These patents collectively contribute to advancements in impact detection and monitoring technologies, particularly in enhancing safety measures for individuals engaged in activities with a risk of head injuries.

Current impact sensors lack the ability to capture both linear acceleration and rotational motion with high accuracy, while maintaining real-time connectivity with mobile applications and cloud-based analytics. The present invention overcomes these limitations by integrating high-G accelerometers, an inertial measurement unit (IMU), real-time clock (RTC) timestamping, and a wireless communication module into a compact wearable device. Therefore, there is a new for improved systems and methods for detecting, recording, and analyzing head impacts that may lead to concussions.

### SUMMARY OF THE INVENTION

The present invention provides a concussion sensor system that captures linear and rotational acceleration data, processes impact events, and transmits the data wirelessly to a mobile application and cloud-based analysis platform.

The invention comprises a dual-sensor integration that combines a high-G accelerometer (±200 g) and an IMU gyroscope for precise motion tracking, Real-Time Wireless Communication that uses Bluetooth Low Energy (BLE) for data transmission, onboard data storage with flash memory that stores impact data when BLE is unavailable that ensures no loss of critical information, event detection & timestamping that detects high-impact events and assigns timestamps for accurate impact history tracking, machine learning & data fusion that uses quaternion-based sensor fusion (e.g., Madgwick filter) to refine impact detection and gravity correction, and a user interface & cloud analysis that displays risk assessments on a mobile app and enables long-term storage and review.

### BRIEF DESCRIPTION OF THE DRAWINGS

A complete understanding of the present invention may be obtained by reference to the accompanying drawing, when considered in conjunction with the subsequent detailed description, in which:

FIG. 1 is a front perspective view of certain embodiments of a concussion sensor;

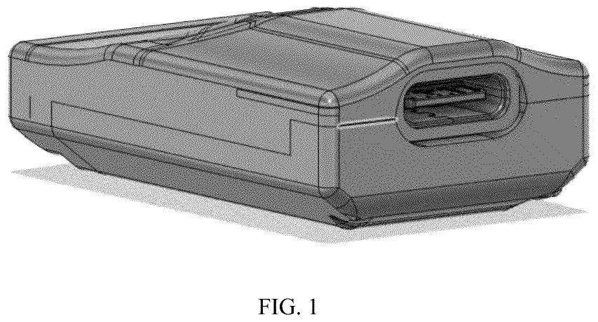

FIG. 2 is a top view of certain embodiments of a concussion sensor;

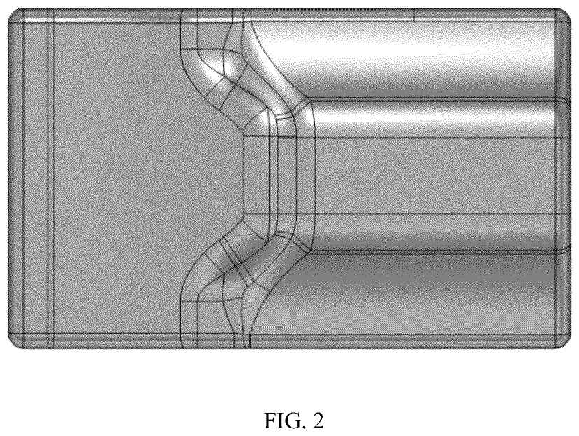

FIG. 3 is a front view of certain embodiments of a concussion sensor;

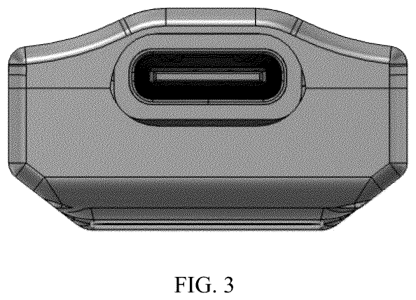

FIG. 4 is a side view of certain embodiments of a concussion sensor;

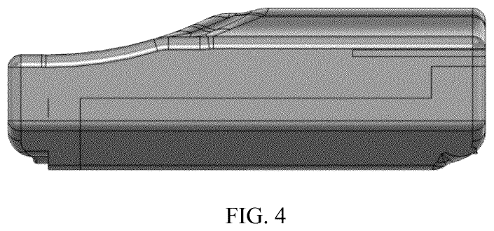

FIG. 5 is a side view of certain internal embodiments of a concussion sensor; and

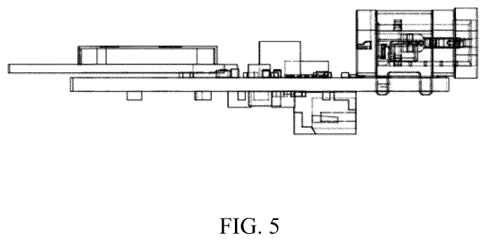

FIG. 6 is a top view of certain internal embodiments of a concussion sensor.

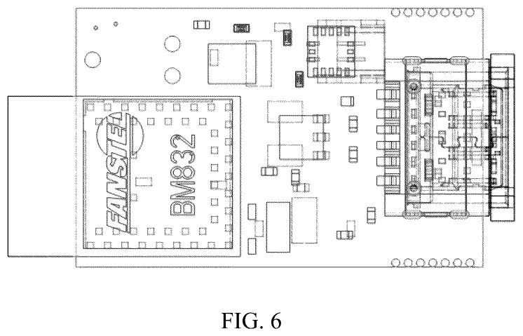

### DETAILED DESCRIPTION

Although the following detailed description contains specific details for the purposes of illustration, those of ordinary skill in the art will appreciate that variations and alterations to the following details are within the scope of the invention. Accordingly, the exemplary embodiments of the invention described below are set forth without any loss of generality to, and without imposing limitations upon, the claimed invention.

Hardware Configuration. Preferred embodiments of the concussion sensor system consist of a single printed circuit board (PCB) containing all core components:

Sensors. High-G Accelerometer (ADXL372) which detects extreme impact forces up to ±200 g. IMU with Gyroscope (ICM-42670-P) that measures lower-range acceleration (±2-16 g) with high precision. Tracks rotational velocities (±125 to ±2000 degrees per second). Provides 16-bit resolution for detailed motion capture.

Microcontroller (MCU). nRF52840 (ARM Cortex-M4 processor with BLE connectivity). Integrated Flash Memory that stores impact data when BLE is unavailable.

Power Management. Lithium-ion or Li—Po battery with USB-C charging. Low-power sleep mode for extended battery life.

Data Storage. RAM Buffering for immediate data storage for high-speed impact event processing. Flash memory backup that retains impact data when BLE transmission is delayed.

Real-Time Clock (RTC). Accurate timestamping of impact events. Synchronization with mobile devices for precise event tracking.

Data Processing & Algorithm. Preferred embodiments of the concussion sensor system consists of the following data processing & algorithm:

Data Acquisition & Event Detection. Impact threshold system that detects events exceeding preset acceleration limits (e.g., 40 g, 50 g, 60 g). Captures pre- and post-impact sensor readings for analysis.

Data Sampling Strategy: High-G accelerometer that records extreme impacts at high-speed. IMU Gyroscope that captures rotational motion for injury risk assessment. Event Windowing that stores impact data before, during, and after the incident. Uses FIFO ring buffer to ensure seamless data capture.

Sensor Fusion Algorithm. Quaternion-Based Fusion (Madgwick Filter) that merges accelerometer and gyroscope data for real-time orientation tracking, corrects for gravity-induced errors, and utilizes impact force normalization that normalizes acceleration data relative to athlete characteristics (height, weight), and computes peak g-force and rotational acceleration metrics.

Machine Learning for Risk Assessment. Concussion risk scoring that analyzes impact patterns across multiple events. Compares user data to historical sports concussion data. Adaptive Thresholds that dynamically adjusts concussion detection sensitivity based on user profile.

Wireless Communication & Mobile Integration. Bluetooth Low Energy (BLE). Real-Time Data Streaming to the mobile application. Low-Energy Mode for extended battery life. Secure Encrypted Data Transfer for HIPAA compliance.

Mobile Application. Displays impact severity and risk analysis. Historical data tracking for long-term monitoring. Emergency alerts for severe impacts.

Server-Side Processing. Stores and analyzes impact data for future injury assessment. Generates Reports for medical personnel and athletic trainers. Integrates with team dashboards for group monitoring.

The invention is a concussion sensor that is able to register impacts surrounding one's head throughout activities.

Certain embodiments comprise a small rectangular sensor, wherein the sensor may contain an accelerometer and a gyroscope. In certain embodiments, the accelerometer may be used to measure linear acceleration on the head throughout activities. Using positive and negative X/Y/Z data inputs, an accurate representation of the head during lateral impacts can be modeled. In certain embodiments, the gyroscope may be used to measure angular acceleration on the head throughout activities. This quantifies the rotational impact on the head throughout an impact. With filters and modeling, an accurate representation of head impacts can be determined.

Using both the accelerometer and the gyroscope allows for a holistic view of forces the head goes through during impact, accounting for both lateral and rotational forces. The weaknesses of each sensor can be applied against each other increasing the strength of the data and its long-term validity. A good example of this is using the relative origin of the gyroscope to account for the drifting nature of the accelerometer, as well as the orientation of the accelerometer to account for the gimbal lock problems within the gyroscope.

Certain embodiments comprise a main circuit board. In certain embodiments, the main circuit board is the brains behind the sensor which allows it to read, write, and process data. The main functionality of the board's motherboard is to process the sensor data to then upload it to the server which it is then analyzed. In beta testing the motherboard used was an Arduino Nano 33 BLE Sense; however, in production models, this is subject to change to more specific parts that fit the needs of the product.

Certain embodiments comprise BLE5.0. In certain embodiments, BLE5.0 is one of the chips in the motherboard that provides Bluetooth 5.0 connectivity. This is what allows the device and the users phone to communicate directly. This chip was built into Arduino Nano sensor mentioned above and will be in the final iteration of the motherboard. It is worth pointing out exclusively due to its critical relevance in the entire system.

Certain embodiments comprise a battery. In certain embodiments, the battery may be a lithium-Ion battery that powers the sensor. Its production model size and shape is yet to be determined. This will most likely be outsourced in the production model.

Certain embodiments comprise LED's. In certain embodiments, there are external LED's that are mounted and visible through the external shell. The initial intent of the LED's is to show a quick glance of the hardest impact sustained since the activity started. Another idea is to have these LED's customizable through the app. The final purpose is yet to be determined but designing them into the final product will allow for more options in the latter stages of product development.

Certain embodiments comprise internal mounts. In certain embodiments, these are the internal mounts for the systems mentioned above. All of these are done in-house in CAD. The material for the final production model will vary, but through rapid prototyping building in PLA was an attractive solution that is worth pursuing. The mounting system must be able to withstand forces up to 200 g, considering that the sensor is responsible for the data.

Certain embodiments comprise an external shell. In certain embodiments, the external shell is the impact proof box that houses all of the systems mentioned above. This has to be crush and puncture resistant considering the types of activities it will be exposed too. The lighter the shell—and entire sensor for that matter—the easier it will be to integrate into the athlete's equipment. This is due to the inertia of the sensor in relation to the athlete. When designing the shell, it could also give a little bit to help destress the sensor during impact. The validity of this data is still sound due to the rigid nature of the internal mounts. A material that is fit for this description is TPU, where it holds a solid shape but depending on the print pattern and angle it can have a lighter or stiffer flex to it. The production model has many possibilities, but a TPU like/hard rubber shell accurately represents that ideology.

In certain embodiments, the sensor itself is a small rectangular device, housing accelerometers and gyroscopes, that registers relative impacts to the head. The sensor would work in parallel with an app that would present all of the data paired with insights surrounding common trends in data. All of the previous data is stored in company servers and made accessible to users. This would allow for a more acute diagnosis when looking back at insights. Depending on the athlete's metrics (body weight, head shape/size) they will get an estimated “g-force value” that will trip the sensor and register the concussion. This will determine the relative grade of concussion on the head, whether it is Mild Traumatic Brain Injury, or Traumatic Brain Injury. This presents the question; how does all of the data get compiled and presented to the user? The raw data right from the accelerometer and the gyroscope will get sent to a company server, this is where the g-force will be analyzed and calculated. Doing these computations over a server means the sensors can have less horsepower, making them smaller and cheaper. The noise on the sensors can be very high depending on the sport. For example, a football player would have high rotational noise when compared to an alpine skier, and a bike rider would have more linear noise than a soccer player. With this, the algorithm used to mitigate noise can be tailored to whatever sport is being played. The weakness of each sensor can be used against each other to help increase the confidence in data increasing its long-term validity. Gyroscopes suffer from gimbal long when used in longer term applications, however the orientation from the accelerometer can be used to help strengthen the gyroscopic data limiting rotational issues long term. Accelerometers can drift and lose their origin point when dealing with noisy data sets, this is where the gyroscope comes in with its relative origin point limiting the accelerometer's drift. There are many applications in software that can help increase confidence in data, many of which, don't require any hardware modifications. It is critical that these filters produce accurate data for the algorithms analyzing the data afterward (garbage in leads to garbage out). After all of these filters are done, there is a solid data set to perform calculations on. In beta testing, g-force can be calculated simply through derivatives. This is how we go from acceleration outputs, to jerk, and then dividing by gravity to get the resultant linear g-force. This process is outlined through the equations below.

$${J}_{x} ( t ) = \frac{d {a}_{x} ( t )}{d t} {J}_{y} ( t ) = \frac{d {a}_{y} ( t )}{d t} {J}_{z} ( t ) = \frac{d {a}_{z} ( t )}{d t}$$

$$J = \sqrt{{J}_{x}^{2} ( t ) + {J}_{y}^{2} ( t ) + {J}_{z}^{2} ( t )} g = \frac{J}{{g}_{0}}$$

The same ideology can be applied to rotational g-forces. This is how we can go from rotational acceleration to velocity, then to centripetal acceleration which is the same resultant as g-forces. This process is outlined through the equations below.

$$a = \frac{Δ w}{Δ t} g = \frac{( a ) ( r )}{{g}_{0}}$$

Keep in mind this was the ideology for the equations in beta testing. Everything above is subject and expected to change for the production model. This is how the sensor ecosystem goes from reading data to a tangible analysis for the user. There are many different external variables that must be considered when testifying to the resultant data. Many measures will have to be taken to ensure correct placement, variables, and vital details are correct before using the product.

All references throughout this application, for example patent documents including issued or granted patents or equivalents; patent application publications; and non-patent literature documents or other source material; are hereby incorporated by reference herein in their entireties, as though individually incorporated by reference, to the extent each reference is at least partially not inconsistent with the disclosure in this application (for example, a reference that is partially inconsistent is incorporated by reference except for the partially inconsistent portion of the reference).

Whenever a range is given in the specification, for example, a temperature range, a time range, or a composition or concentration range, all intermediate ranges and subranges, as well as all individual values included in the ranges given are intended to be included in the disclosure. It will be understood that any subranges or individual values in a range or subrange that are included in the description herein can be excluded from the claims herein.

All patents and publications mentioned in the specification are indicative of the levels of skill of those skilled in the art to which the invention pertains. References cited herein are incorporated by reference herein in their entirety to indicate the state of the art as of their publication or filing date and it is intended that this information can be employed herein, if needed, to exclude specific embodiments that are in the prior art. For example, when compositions of matter are claimed, it should be understood that compounds known and available in the art prior to Applicant's invention, including compounds for which an enabling disclosure is provided in the references cited herein, are not intended to be included in any composition of matter claims herein.

As used herein, “comprising” is synonymous with “including,” “containing,” or “characterized by,” and is inclusive or open-ended and does not exclude additional, unrecited elements or method steps. As used herein, “consisting of” excludes any element, step, or ingredient not specified in the claim element. As used herein, “consisting essentially of” does not exclude materials or steps that do not materially affect the basic and novel characteristics of the claim. In each instance herein any of the terms “comprising”, “consisting essentially of,” and “consisting of” may be replaced with either of the other two terms. The invention illustratively described herein suitably may be practiced in the absence of any element or elements, limitation or limitations which is not specifically disclosed herein.

The terms “first,” second,” “top,” “bottom,” etc., as used herein, are intended for illustrative purposes only and do not limit the embodiments in any way. Additionally, the term “plurality,” as used herein, indicates any number greater than one, either disjunctively or conjunctively, as necessary, up to an infinite number. Further, “Providing” an article or apparatus, as used herein, refers broadly to making the article available or accessible for future actions to be performed on the article, and does not connote that the party providing the article has manufactured, produced, or supplied the article or that the party providing the article has ownership or control of the article.

One of ordinary skill in the art will appreciate the art within the drawings and can employ various alterations to the design within those presented. The drawings are exemplary of a design and are illustrative of the invention but should not be construed to create limitations of the invention. The invention in the drawings suitably may be practiced in variations without departure from the spirit of the invention.

One of ordinary skill in the art will appreciate that starting materials, biological materials, reagents, synthetic methods, purification methods, analytical methods, assay methods, and biological methods other than those specifically exemplified can be employed in the practice of the invention without resort to undue experimentation. All art-known functional equivalents, of any such materials and methods are intended to be included in this invention. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

Since other modifications and changes varied to fit particular operating requirements and environments will be apparent to those skilled in the art, the invention is not considered limited to the example chosen for purposes of disclosure and covers all changes and modifications which do not constitute departures from the true spirit and scope of this invention.

Having thus described the invention, what is desired to be protected by Letters Patent is presented in the subsequently appended claims.

What is claimed is:

1. A concussion sensor system comprising:
an accelerometer for detecting impact forces and impact events;
a gyroscope for measuring rotational acceleration;
a microcontroller for real-time communication;
an onboard memory for local data storage;
a real-time clock for timestamping sensor readings;
a mobile application for receiving, processing, and displaying concussion risk assessments.

2. The system of claim 1, further comprising:
a quaternion-based sensor fusion algorithm for filtering and correcting motion data;
a machine learning analysis for adaptive concussion risk scoring; and
a cloud-based data storage for long-term tracking.

3. The system of claim 1, wherein:
impact events are detected based on predefined linear and rotational acceleration thresholds.

4. The system of claim 1, wherein:
the sensor dynamically switches between low-power and active modes based on motion detection.

5. The system of claim 1, wherein:
the microcontroller uses a Bluetooth low energy for real-time communication.

6. The system of claim 1, wherein:
the mobile app provides real-time concussion risk assessments and alerts.

7. The system of claim 1, wherein:
the system integrates with team dashboards for multi-player monitoring.
